(19) United States
(12) Patent Application Publication
(10) Pub. No.: US 2009/0155395 A1
(43) Pub. Date: Jun. 18, 2009

(54) SF303T, Anti-Inflammatory and Anti-Acne Agent

(75) Inventor: Suying Liu, Belmont, CA (US)

Correspondence Address: WPAT, PC;INTELLECTUAL PROPERTY ATTORNEYS, 2030 MAIN STREET, SUITE 1300, IRVINE, CA, 92614, US

(73) Assignee: SAGITTARIUS LIFE SCIENCE CORP., Taipei Shien (TW)

(21) Appl. No.: 11/955,889

(22) Filed: Dec. 13, 2007

(51) Int. Cl. A61K 36/00; A61K 36/538; A61P 17/10; A61P 17/00

(52) U.S. Cl. 424/745; 424/725

(57) ABSTRACT

The present invention provides a composition comprising Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora and Glycyrrhizae radix for treating or prophylaxis of acne and inflammation related to skin disorders.

### FIELD OF THE INVENTION

This present invention relates to a composition and a method for treating or prophylaxis acne and inflammation related to skin disorders.

### BACKGROUND OF THE INVENTION

Acne is a physiological and clinical disease of pilosebaceous unit in face, chest and back. It affects between 40 and 50 million people in the United States. Although acne affects adolescents mainly, it also affects children and adults. Clinical acne persists into middle age in 12% of women and 3% of men. In 10-12 years old, 28 to 61% have acne, and 16-18 years old, 79 to 95% have acne (Cordain L., et. al., Arch Dermatol. 2002; 138: 1584-1590). Depressed or hypertrophic scar formation due to acne affects some proportion of individuals. The effects of acne on pain/discomfort, anxiety/depression, and psychosocial disability are tremendous. There is a deficit and a huge market demand for a potent anti-acne agent with low side effects.

Acne vulgaris is a common, inflammatory disease of the pilosebaceous duct. Its etiology is multifactorial in nature (Purdy S and deBerker D. BMJ 2006; 333: 949-953). One of the etiologies of acne is hypercornification of sebaceous follicles that lead to plugging of follicles. In normal conditions, the cornified layer of the follicle remains thin by constant desquamation. When there is disorder in desquamation processes, the ductal epithelium thickens and the ductal lumen becomes narrow. The process of ductal hypercornification causes the formation of a microcomedone that may evolve into either a comedone or an inflammatory lesion.

Androgen stimulation is an important factor in adolescent and adult acne. Androgen causes an increased production of sebum (Thiboutot D. J. Invest. Dermatol. 2004; 123(1): 1-12). At puberty, the gonadal and adrenal glands mature and secrete increased amount of androgen to increase the activity of sebaceous glands and produce more sebum. Males produce 10 times as much androgen (from both adrenal gland and testis) as females, so that more males develop severe cases of acne. Some patients display acne without higher levels of circulating androgen, this condition may result from increased androgen receptor-to-ligand affinity, increased 5-α-reductase activity, or increased numbers of androgen receptors at the pilosebaceous unit in the skin. Studies have shown that higher rates of testosterone conversion to dihydrotestosterone by type I steroid 5-α-reductase exist in acne-bearing skin when compared with non-acne-bearing skin (Chen W C., et. al., J. Invest. Dermatol. 2002; 119: 992-1007).

A skin resident anaerobic microorganism, Propionibacterium acnes colonizes in sebum, produces chemotactic factors that results in the production of inflammatory mediators and reactive oxygen species which contribute to the inflammatory reaction in papulopustular acne lesion. The activities of superoxide dismutase (SOD), glutathione peroxidase (GSH-Px) were significantly decreased in the leukocytes of acne patients, and catalase (CAT) enzymes as well as the level of thiobarbituric acid reactive substance (TBARS) were higher in the leukocytes of acne patients than normal controls. Anti-oxidative defense enzymes are impaired in papulopustular acne, and anti-oxidative agents may be valuable in treatment (Basak P Y., et. al., J. Dermatol. 2001; 28(3): 123-127).

Acne may also be exacerbated by chemicals and environmental irritants, such as halogenated aromatic hydrocarbons (English JSC, British Medical Bulletin. 2003; 68:129-142), and hot humid environments. There were some evidences to show that genetic factors influence the susceptibility to acne in adolescence (Bataille V, et. al., J. Invest. Dermatol. 2002; 119: 1317-1322). The data available did not show significant correlation of variable diets with acne. Hormonal profile and 5-α-reductase type I have some correlation with incidence of acne.

A variety of medications with various effectiveness and side effects are available for the treatment of acne. Most commonly used topical agents are Tretinoin, Adapalene, Azelaic acid, Isotretinoin, Benzoyl peroxide and antibiotics. Oral agents contain antibiotics, hormone, and Isotretinoin. Some topical agents cause skin irritation and dermatitis, and some cause photosensitivity. Antibiotics induce drug resistant and cross-resistant strains of bacteria. Hormonal therapy (anti-androgen) is effective in patients with endocrine abnormalities only. Isotretinoin is teratogenic, and with many other adverse effects (Haider A., and Shaw, J C. JAMA. 2004; 292(6): 726-735. Gollnick H. Drugs. 2003; 63(15): 1579-1596. Raudrant D. and Rabe T. Drugs. 2003; 63(5): 463-492).

### SUMMARY OF THE INVENTION

The present invention provides a composition, SF303T, comprising Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora and Glycyrrhizae radix.

The present invention also provides a method for treating or prophylaxis of acne and inflammation related to skin disorders of a subject, administrating the composition of the present invention to the subject.

### BRIEF DESCRIPTION OF THE DRAWINGS

FIG. 1. Average percent changes of overall improvement in acne scores. The score, represented as the percentage of improvement over baseline, was derived by globally considering the severity and number of the lesions.

### DETAIL DESCRIPTION OF THE INVENTION

The present composition, SF303T, shows activity on treatment and prevention the recurrence of acne, skin disorders caused by reactive oxygen species and inflammation, and relieving symptoms of pain and inflammation associated with acne and other skin disorders in both male and female after intake for 4 to 8 weeks. Since it does not have direct anti-bacterial activity against Propionibacterium acnes in vitro, there is no concern on the development of drug resistance by antibiotics type of medications. It is well documented that increase in active form of testosterone during puberty increases sebum production. The special fatty acids and environment of sebum favor the growth of P. acne. SF303T has moderate activity on inhibition of steroid 5-α-reductase with an IC₅₀ of about 100 μg/ml. The inhibition of steroid 5-α-reductase leads to lower formation of dihydrotestosterone, the active form of testosterone, thus, less sebum produced. Less sebum, less colonization of P. acne, less chemotactic and inflammatory mediators released, less acnes formed. SF303T also slightly inhibited COX I, and moderately inhibited COX II, which resulted in anti-inflammation and pain relieving activities. SF303T also has SOD mimetic activity to neutralize ROS that attacks skin tissues. According to the traditional Chinese medicinal practice, the causes of acne are the building up of heat in the blood, and ascending of evil wind to express on the skin of face, chest and back. The combination of these herbs in SF303T is designated to cool and clear the heat in the blood, to promote circulation of qi and blood, and to clear the dampness and evil wind. The in vitro activities of anti-inflammatory (COX I and II inhibition), anti-sebum formation (steroid 5-α-reductase inhibition), anti-reactive oxygen species (SOD mimetic activity) effects of SF303T composition are evident in human use experiences on treatment and prevention the recurrence of acne, skin disorders caused by reactive oxygen species and inflammation, and relieving symptoms of pain and inflammation associated with acne and other skin disorders.

Accordingly, the present invention provides a composition, SF303T, for treating or preventing the recurrence of acne, skin disorders caused by reactive oxygen species and inflammation, and relieving symptoms of pain and inflammation associated with acne and other skin disorders is composed of the extract from the whole plant or a part of the plant of Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora, and Glycyrrhizae radix.

The present composition further comprises a pharmaceutically or food acceptable excipient, carrier or diluent to various dosage form by skills of the known prior arts to mask the bitter flavor.

In a preferred embodiment of the invention, the weight-to-weight ratio of Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora, and Glycyrrhizae radix is 2.5˜10:2˜8:1.5˜6:1.25˜5:1.25˜5:1.25˜5:1.125˜4.5:1˜4:0.75˜3:0.75˜3:0.75˜3:0.5˜2. In a more preferred embodiment of the invention, the weight-to-weight ratio is 5:4:3:2.5:2.5:2.5:2.25:2:1.5:1.5:1.5:1.

The present composition has anti-steroid 5-α-reductase, anti-cyclooxygenase I (COX I), anti-cyclooxygenase II (COX II) or superoxide dismutase (SOD) mimetic activity. Thus, the present composition manifests the anti-dihydrotestosterone formation, anti-inflammation or anti-reactive oxygen species activities for treating and preventing the recurrence of acne, skin disorders caused by oxidation insults and inflammation, and relieving symptoms of pain and inflammation associated with acne and other skin disorders.

The present composition could be in a form of lozenge, tablet, film coated tablet, capsule, soft gel capsule, granule, powder, pill, solution, emulsion, injection solution, injection, ointment, cream, lotion, serum, spray or inhalant.

The present invention also provides a method for treating or prophylaxis acne to a subject, administrating the composition of the present invention to the subject. The weight-to-weight ratio of composition is 2.5˜10:2˜8:1.5˜6:1.25˜5:1.25˜5:1.25˜5:1.125˜4.5:1˜4:0.75˜3:0.75˜3:0.75˜3:0.5˜2. The composition is administered by oral, intramuscular injection, intra-venous injection, subcutaneous injection, mucosal membrane or topical routes.

### EXAMPLES

### Example 1

### Herbal Composition and Manufacture Processes

The composition of the present invention, SF303T, comprised of Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora, and Glycyrrhizae radix at the weight to weight ratio of 2.5˜10:2˜8:1.5˜6:1.25˜5:1.25˜5:1.25˜5:1.125˜4.5:1˜4:0.75˜3:0.75˜3:0.75˜3:0.5˜2. The most favorable ratio was 5:4:3:2.5:2.5:2.5:2.25:2:1.5:1.5:1.5:1.

1. The Decoction of SF303T

The combined herbs at the favorable ratio could be made into decoction with 5 to 10 folds of water at 85 to 100□ for 2 to 4 hours.

2. The Extract Powder of SF303T

The components could be soaked in 5 to 10 folds of deionized water (by weight) at room temperature for 2 hours and then extracted at 85 to 100□ for 2 to 4 hours twice. The combined extracted solution was concentrated under vacuum at 55 to 60□ until the relative density was 1.05 to 1.10, and then was spray dried. The yield of the extracted powder was about 25 to 35% of the raw material.

The resulting herbal powder could be incorporated into lozenge, tablet, film coated tablets, capsule, soft gel capsule, granule, powder, pill, solution, emulsion, injection solution, injection, ointment, cream, lotion, serum, spray, inhalant, or other pharmaceutically acceptable forms for various administration routes. The daily effective dosage was 3.5 to 5 grams of raw material or the equivalent extract powder.

### Example 2

### Biological Activities

1. Propionibacterium acnes

Minimum inhibitory concentration (MIC) was determined by the broth dilution method (Edwards, J. R. et. al., Antimicrob Agents Chemother. 1989; 33(2):215-22).

SF303T was dissolved in 100% DMSO and serially diluted in solvent to desired stock concentrations. 10 ul aliquot (1% DMSO final concentration) of serial dilutions were added into 0.99 ml of Reinforced Clostridial Medium (Oxiod, England.) with 1-5×10⁵ CFU/ml of Propionibacterium acnes (ATCC 6919). The plates were incubated at 37° C. for 48 hours under anaerobic condition (Mixed gas N₂ 85% and CO₂ 15%) and then visually examined and scored positive (+) for inhibition of growth or turbidity, or negative (−) for no effect upon growth or turbidity. Vehicle-control and active reference agents were used as blank and positive controls, respectively.

TABLE 1
The inhibition of Propionibacterium acnes by SF303T

| Treatment | MIC (μg/ml) |
|---|---|
| Ampicillin | 0.1 |
| Cephalothin | — |
| *Gentamicin | 30 |
| Ofloxacin | 1 |
| Tetracycline | 1 |
| Vancomycin | 1 |
| SF303T | >100 |

*Indicates standard reference agent used.

Propionibacterium acnes were the major bacteria resident on skin and related to acne. As shown in Table 1, SF303T did not have direct anti-bacterial activity up to 100 μg/ml in vitro.

2. Steroid 5-α-reductase

Steroid 5-α-reductase isolated from liver of Wistar rat by conventional centrifugation was used (Liang T, et. al., Endocrinology. 1985;117(2): 571-9).

SF303T or vehicle was incubated with 20 μg/ml steroid 5-α-reductase preparation containing 1 μM testosterone and 50 μM NADPH in DTT buffer, pH 6.5, at 37° C. for 30 minutes. The reaction was terminated by addition of 1 N HCl and neutrilized by 1 N NaOH. Testosterone was quantitated by using a Testosterone EIA Kit.

TABLE 2
The inhibition of steroid 5-α-reductase by SF303T

| Treatment | IC₅₀ (μg/ml) |
|---|---|
| *Finasteride | 0.0093 |
| γ-Linolenic Acid | 3.8980 |
| SF303T | ~100 |
| | (at 100 μg/ml inhibited 44%) |

*Indicates standard reference agent used.

The local conversion of testosterone to the more potent androgen dihydrotestosterone (DHT) by 5-α-reductase played a role in sebum production. Two distinct isozymes were found. In human, Type I 5-α-reductase was predominant in the sebaceous glands of most regions of the skin, including scalp and liver, and was responsible for approximately one-third of circulating DHT. Type II 5-α-reductase was primarily found in prostate, seminal vesicles, epididymis, and hair follicles as well as liver, and was responsible for two-thirds of circulating DHT.

As shown in Table 2, SF303T moderately inhibited steroid 5-α-reductase with an IC₅₀ of about 100 μg/ml (at 100 μg/ml inhibited 44%).

3. COX I Inhibition

Human platelet COX I (COX; prostaglandin endoperoxide synthase, EC 1.14.99.1) was used (Chan C C, et. al., JPET 1999; 290:551-560. Swinney D C, et. al., J Biol Chem. 1997; 272: 393-398).

SF303T or vehicle was incubated with human platelets (5×10⁷/ml) containing the phospholipase inhibitor methyl linolenyl fluorophosphonate (MLnFP) (100 μM) for 15 minutes at 37° C. Arachidonic acid (100 μM) was then added and incubated for a further 15 minutes. The reaction was stopped by addition of 1 N HCl and neutralized with 1 N NaOH. PGE₂ levels in the supernatant were determined using the Amersham EIA kit.

TABLE 3
The inhibition of COX 1 by SF303T

| Treatment | IC₅₀ (μg/ml) |
|---|---|
| Aspirin | 0.757 |
| Celecoxib | >3.814 |
| Diclofenac | 0.011 |
| *Indomethacin | 0.016 |
| Niflumic acid | >2.822 |
| Nimesulide | >3.144 |
| Rofecoxib | >3.038 |
| SF303T | >100 |
| | (at 100 μg/ml inhibited16%) |

*Indicates standard reference agent used.

Cyclooxygenase catalyzes the conversion of arachidonic acid to form the cyclic endoperoxides (PGG and PGH) and subsequent metabolite products including prostaglandins and thromboxanes. There are two isoforms of this enzyme, cyclooxygenase-1 and -2 (COX-1 and COX-2). COX-1 was constitutively expressed in most cells. In contrast, COX-2 was not normally present but could be induced by certain serum factors, cytokines, growth factors and endotoxin. Inhibitors of COX-1 exhibited antithrombotic activity and might also cause gastric ulceration. Inhibitors of COX-2 exhibited anti-inflammatory activity and might inhibit some mitogenic actions.

As shown in Table 3, SF303T slightly inhibited COX I at 100 μg/ml.

4. COX II Inhibition

Human recombinant Cyclooxygenase-2 expressed in Sf21 cells (Sigma, C-0858) was used (Riendeau D, et. al., Can J Physiol Pharmacol. 1997; 75(9):1088-95. Warner J D, et. al., Proc. Natl. Acad. Sci. 1999; 96: 7563-68).

SF303T or vehicle was pre-incubated with 0.11 U enzyme, 1 mM reduced GSH, 500 μM phenol and 1 μM hematin in Tris-HCl pH 7.7 at 37° C. for 15 minutes. The reaction was initiated by addition of 0.3 μM arachidonic acid for 5 minutes and terminated by further addition of 1 N HCl. Following centrifugation, substrate conversion to PGE₂ was measured by an Amersham EIA kit.

TABLE 4
Inhibition of COX II by SF303T

| Treatment | IC₅₀ (μg/ml) |
|---|---|
| Aspirin | 106.294 |
| Celecoxib | 0.005 |
| Diclofenac | 0.022 |
| Indomethacin | 0.143 |
| Niflumic acid | 0.195 |
| *Nimesulide | 0.053 |
| Rofecoxib | 0.308 |
| SF303T | ~100 |
| | (at 100 μg/ml inhibited 45%) |

*Indicates standard reference agent used.

As shown in Table 4, SF303T moderately inhibited COX II with an IC₅₀ of about 100 μg/ml (at 100 μg/ml inhibited 45%).

5. Free Radical Scavenger, Superoxide Dismutase (SOD) Mimetic Activity

The method to measure SOD (SOD, EC.1.15.1.1) activity was based on the inhibition of nitroblue tetrazolium reduction with xanthine-xanthine oxidase used as a superoxide generator (Sun Y, et. al., Clin. Chem. 1988; 34: 497-500).

SF303T or vehicle was incubated with 0.12 mM xanthine, 6 mU xanthine oxidase, 27 μM nitroblue tetrazolium (NBT), 0.11 mM EDTA, 0.005% bovine serum albumin and Na₂CO₃ at pH 10.5 at 25° C. for 20 minutes. Conversion of xanthine to uric acid +O⁻+NBT to formazan was then determined by measurement of absorbance at 595 nm and percent inhibition by superoxide dismutase mimetic or test compound was calculated.

TABLE 5
SOD mimetic activity of SF303T

| Treatment | IC₅₀ (μg/ml) |
|---|---|
| (−)-Epicatechin gallate | 2.698 |
| *Superoxide dismutase (SOD) | 0.475 |
| SF303T | 100 |

*Indicates standard reference agent used, Human SOD MW = 2 × 28.3 KDa.

Reactive oxygen species (ROS: O₂⁻, H₂O₂ and OH⁻) played an important role in pulmonary oxygen toxicity, inflammation, ischemia/reperfusion injury, aging etc., and that superoxide dismutase provided a defense against superoxide radicals. SOD catalyzed the dismutation of two superoxide radicals (O₂⁻) into O₂ and H₂O₂. As shown in Table 5, SF303T had SOD mimetic activity with an IC₅₀ of 100 μg/ml.

6. Human Use Experiences:

A. Acne was Classified into Three Types:
(1) Comedonal acne (non-inflammatory): It was caused by plugging of abnormally cornified cells of the pilosebaceous duct in the dilated follicle.
   Whitehead: a dilated hair follicle filled with keratin, sebum and bacteria with an obstructed opening to the skin.
   Blackhead: a dilated hair follicle filled with keratin, sebum and bacteria with a wide opening to the skin capped with a blackened mass of skin debris.
(2) Papulo-pustular acne (inflammatory): The lesion was the result of the inflammatory response to Propionibacterium acnes in pilosebaceous duct.
   Papule: small bump less than 5 mm in diameter.
   Pustule: small bump with a visible central core of purulent material.
(3) Nodular acne (inflammatory): The lesion was the result of the inflammatory response to Propionibacterium acnes in pilosebaceous duct.
   Nodule: bump greater than 5 mm in diameter.

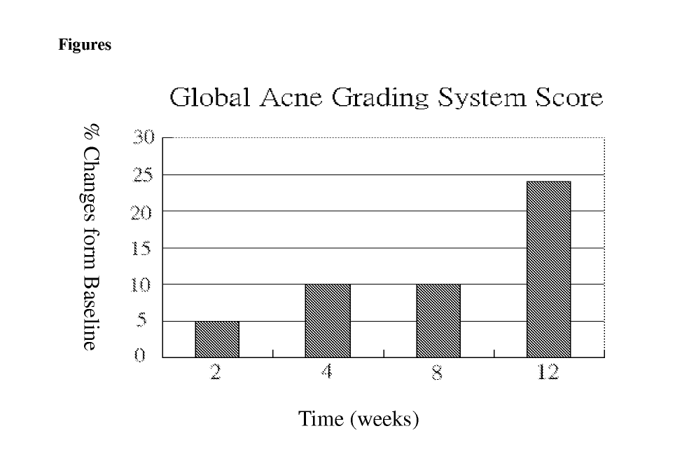

B. The Human Experiment
(1) The decoction of SF303T
   Twelve healthy volunteers, 3 males and 9 females, age 18 to 42, exhibiting moderate to severe acne, were assessed for their severity and counts of acne according to the Global Acne Grading System (Doshi, et. al., International J of Dermatology. 1997; 36(6): 416-418) before and after usage of decoction of SF303T raw material at 3.5 grams per day for 12 weeks periodically.
   Global Acne Grading System (Combined Acne Severity Classification):
   0=Normal, clear skin with no evidence of acne vulgaris.
   1=Skin was almost clear: rare non-inflammatory lesions present, with rare non-inflamed papules (papules must be resolving and may be hyperpigmented, though not pink red).
   2=Some non-inflammatory lesions were present, with few inflammatory lesions (papules/pustules only; no nodulo-cystic lesions).
   3=Non-inflammatory lesions predominate, with multiple inflammatory lesions evident: several to many comedones and papules/pustules, and there may or may not be one small nodulo-cystic lesion.
   4=Inflammatory lesions are more apparent: many comedones and papules/pustules, there may or may not be a few nodulo-cystic lesions.
   5=Highly inflammatory lesions predominate: variable number of comedones, many papules/pustules nodulo-cystic lesions.
   The results as shown in FIG. 1, the decoction of SF303T showed an overall improvement of 24% by global acne grading system comparing to the baseline.
(2) The extract powder of SF303T
   One hundred and six healthy volunteers, 47 males and 59 females (placebo group: male/female=21/32; SF303T group: male/female=26/27), age 17 to 24 (placebo group: 20.36±2.68; SF303T group: 20.51±2.53), with lesion counts between 21.89±10.18 (placebo group) and 21.36±13.15 (SF303T group) and severity score between 2.66±1.07 (placebo group) and 2.60±1.04 (SF303T group) were randomized blindly into two groups: placebo and SF303T extract powder 1.2 grams/day for 4 weeks.
   Lesion count: Count the number of facial closed comedones (white heads), open comedones (black heads), papules, pustules, and nodules at the baseline and 4 weeks after treatment.
   Severity assessment
   Slight: some blackhead comedones, no inflammatory lesion (Score: 1).
   Mild: some comedones with some papule inflammatory lesions limited on facial area (Score: 2).
   Moderate: more comedones with more papule, pustule inflammatory lesions distributed on face, neck and upper abdomen and back (Score: 3).
   Severe: nodules and inflammatory scar lesions distributed over the upper body (Score: 4).

TABLE 6
Comparisons of acne number counts and lesion severity scores of placebo and SF303T
extract powder treatment for 4 weeks

| | Placebo control (n = 53) | | SF303T-extract powder (n = 53) | |
|---|---|---|---|---|
| Item | Before | After | Before | After |
| Closed comedones | 11.85 ± 6.77 | 11.57 ± 7.08 | 11.32 ± 7.81 | 9.09 ± 6.35 |
| (Whiteheads) | | | | |
| Open comedones | 5.36 ± 3.05 | 5.30 ± 3.17 | 5.45 ± 2.97 | 4.13 ± 2.48 |
| (Blackheads) | | | | |
| Papules | 2.11 ± 1.46 | 2.09 ± 1.50 | 2.08 ± 1.64 | 1.62 ± 1.60 |
| Pustules | 1.08 ± 1.03 | 1.15 ± 1.01 | 1.13 ± 1.29 | 0.75 ± 1.14 |
| Nodules | 1.49 ± 0.95 | 1.44 ± 1.08 | 1.38 ± 0.87 | 0.85 ± 0.72 |
| Total acne count | 21.89 ± 10.08 | 21.55 ± 11.44 | 21.36 ± 13.15 | 16.45 ± 0.16*^{#} |
| Decrease in | | 0.34 ± 3.82 | | 4.91 ± 5.88^{##} |
| acne count | | | | |
| Decrease in % | | 1.60 ± 18.62 | | 20.80 ± 25.86^{###} |
| Lesion severity | 2.66 ± 1.07 | 2.62 ± 0.95 | 2.60 ± 1.04 | 2.08 ± 0.96*⁺ |
| Decrease | | 0.04 ± 0.39 | | 0.53 ± 0.61^{##} |
| in severity | | | | |

*Compared to before treatment, P < 0.01;
^{#}Compared to control, P < 0.05;
⁺Compared to control, P < 0.01;
^{##}Compared to control, P < 0.01;
^{###}Compared to control, P < 0.01

Compared to placebo, the SF303T extract powder group had significantly lower total number of acne lesions (placebo: 21.89±10.08 to 21.55±11.44; SF303T: 21.36±13.15 to 16.45±10.16 at 4 weeks, P<0.01), and less severity in lesion scores (placebo: 2.66±1.07 to 2.62±0.95; SF303T: 2.60±1.04 to 2.08±0.96 at 4 weeks, P<0.01) after 4 weeks of treatment as analyzed by paired t Test, t Test or X² analysis.

Safety: There were no significant differences in hematology, blood chemistry, urine analysis and vital signs between the two groups before and after 4 weeks treatment of SF303T.

It will be readily apparent to a person skilled in the art that varying substitutions and modifications may be made to the invention disclosed herein without departing from the scope and spirit of the invention. All patents and publications mentioned in the specification are indicative of the levels of those of ordinary skill in the art to which the invention pertains.

1. A composition comprising Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora and Glycyrrhizae radix.

2. The composition of claim 1, which further comprises a pharmaceutically or food acceptable excipient, a carrier or a diluent.

3. The composition of claim 1, wherein the Rehmanniae radix, Paeonia suffruticosa, Saposhnikovia divaricata, Schizonepeta multifida, Saphora flavescens, Paeonia lactiflora, Dictamnus dasycarpus, Mentha arvensis, Rheum palmatum, Xanthium sibiricum, Campsis grandiflora, Glycyrrhizae radix are presented respectively in dried, weight-to-weight ratios of 2.5˜10:2˜8:1.5˜6:1.25˜5:1.25˜5:1.25˜5:1.125˜4.5:1˜4:0.75˜3:0.75˜3:0.75˜3:0.5˜2.

4. The composition of claim 3, wherein the weight-to-weight ratio is 5:4:3:2.5:2.5:2.5:2.25:2:1.5:1.5:1.5:1.

5. The composition of claim 1, which has anti-steroid 5-α-reductase, anti-cyclooxygenase I (COX I), anti-cyclooxygenase II (COX II) or superoxide dismutase (SOD) mimetic activity.

6. The composition of claim 1, which has anti-dihydrotestosterone formation, anti-inflammation or anti-reactive oxygen species activities.

7. The composition of claim 1, which is in a form of lozenge, tablet, film coated tablet, capsule, soft gel capsule, granule, powder, pill, solution, emulsion, injection solution, injection, ointment, cream, lotion, serum, spray or inhalant.

8. A method for treating or prophylaxis acne to a subject, administrating the composition of claim 1 to the subject.

9. The method of claim 8, wherein the weight-to-weight ratio of composition is 2.5˜10:2˜8:1.5˜6:1.25˜5:1.25˜5:1.25˜5:1.125˜4.5:1˜4:0.75˜3:0.75˜3:0.75˜3:0.5˜2.

10. The method of claim 8, wherein the composition is administered by oral, intramuscular injection, intra-venous injection, subcutaneous injection, mucosal membrane or topical routes.

11. The method of claim 8, which is in a form of lozenge, tablet, film coated tablet, capsule, soft gel capsule, granule, powder, pill, solution, emulsion, injection solution, injection, ointment, cream, lotion, serum, spray or inhalant.
